(19) United States
(12) Patent Application Publication
(10) Pub. No.: US 2026/0183152 A1
(43) Pub. Date: Jul. 2, 2026

(54) METHOD OF PRODUCING BOXER-STYLE PANT DISPOSABLE ABSORBENT ARTICLE

(71) Applicant: The Procter & Gamble Company, Cincinnati, OH (US)

(72) Inventors: Michael Devin LONG, Harrison Township, OH (US); Abhishek Prakash SURUSHE, Kelkheim (DE); Uwe SCHNEIDER, Cincinnati, OH (US)

(21) Appl. No.: 19/549,076

(22) Filed: Feb. 25, 2026

Related U.S. Application Data
(63) Continuation of application No. PCT/US2023/074727, filed on Sep. 21, 2023.

(51) Int. Cl. A61F 13/15; A61F 13/496

(52) U.S. Cl. CPC A61F 13/15707; A61F 13/496; A61F 2013/15715

(57) ABSTRACT

Methods for producing a boxer-style absorbent article, the methods including bonding a plurality of substrates with a discrete inseam bond to form a cover assembly; unfolding the cover assembly at an apex of the discrete inseam bond; and bonding an absorbent assembly to the unfolded cover assembly over the discrete inseam bond in a position providing contact between the absorbent assembly and the apex of the discrete inseam bond. Boxer-style absorbent articles having a reduced distance between an inseam bond and an absorbent assembly.

### CROSS REFERENCE TO RELATED APPLICATION

The present application is a continuation of patent application number PCT/US2023/074727 filed on Sep. 21, 2023, which is incorporated herein by reference.

### FIELD

The present disclosure relates generally to boxer-style pant disposable absorbent articles and more specifically to methods of making such articles.

### BACKGROUND

A boxer-style pant disposable absorbent article/diaper is desirable primarily for older children struggling with nighttime incontinence—where this form of product can offer consumers a more garment-like fit and appearance. It has been discovered that prior art methods of forming such absorbent articles result in undesirably excessive distance between an absorbent assembly disposed within the absorbent article and an inseam bond, leading to ill-fitting and uncomfortable garments. It has been discovered that prior art methods of producing boxer-style absorbent articles require tolerancing built into the placement of the inseam bond versus the folded bottom edge of the absorbent assembly to account for web tracking between the time the pad is bi-folded and the time when an inseam bond is applied. As a result of this tolerance error, if the inseam bond were to contact the folded chassis edge, this would damage the product or render the inseam bond unusable. Hence, prior art methods, require undesirable distance between the absorbent assembly and inseam bond locations. A need, therefore, exists not only for boxer-style absorbent articles with a reduced distance between the absorbent assembly disposed and the inseam bond, but also for methods of producing such articles. The discussion of shortcomings and needs existing in the field prior to the present disclosure is in no way an admission that such shortcomings and needs were recognized by those skilled in the art prior to the present disclosure.

### SUMMARY

Various embodiments solve the above-mentioned problems and provide methods useful for producing boxer-style absorbent articles having a reduced distance between an inseam bond and an absorbent assembly. To solve for the issue of a required gap between the chassis edge and inseam bond, the methods of the present disclosure employ a unique order of operations and introduces new process steps. According to the prior art, an inseam bond is created after the absorbent assembly is transferred and attached to the receiving belts, which requires additional clearance between the inseam bond and the absorbent assembly to avoid damaging the absorbent assembly when applying the inseam bond. On the other hand, the methods according to various configurations create an inseam bond before the chassis is ever attached to the receiving belts. The methods for producing a boxer-style absorbent article may include bonding a plurality of substrates with a discrete inseam bond to form a cover assembly; unfolding the cover assembly at an apex of the discrete inseam bond; and bonding an absorbent assembly to the unfolded cover assembly over the discrete inseam bond in a position providing contact between the absorbent assembly and the apex of the discrete inseam bond.

A method may be provided for assembling an absorbent article. The method may comprise advancing a first substate in a machine direction. The first substrate may comprise a first surface and an opposing second surface, and a first edge separated from a second edge in a cross direction. The method may further comprise advancing a second substate in the machine direction. The second substrate may comprise a first surface and an opposing second surface, and a first edge separated from a second edge in a cross direction. The method may further comprise positioning the second surface of the first substrate in a facing relationship with the first surface of the second substrate and forming a cover assembly by bonding the first substate and the second substrate together adjacent the first edge of the first substrate and the first edge of the second substrate with a discrete inseam bond. Next, the method may comprise unfolding the cover assembly along the discrete inseam bond such that the second edge of the first substrate is separated from the second edge of the second substrate in the cross direction and positioning an absorbent assembly on the unfolded cover assembly and over the inseam bond. Finally, the method may comprise folding the cover assembly and absorbent assembly along the inseam bonds to position the second surface of the first substrate in a facing relationship with the first surface of the second substrate; bonding the first substrate and the second substrate of the folded cover assembly together with side seam bonds on opposing sides of the folded absorbent assembly; and cutting the folded cover assembly along the side seam bonds to form a discreate absorbent article.

According to various other configurations, a method for producing a boxer-style absorbent article may comprise bonding a plurality of substrates with a discrete inseam bond to form a cover assembly; cutting along the discrete inseam bond to separate an inseam scrap portion from the cover assembly; unfolding the cover assembly at an apex of the discrete inseam bond; bonding an absorbent assembly to the unfolded cover assembly over the discrete inseam bond in a position providing contact between the absorbent assembly and the apex of the discrete inseam bond; refolding the cover assembly and the absorbent assembly at the apex of the discrete inseam bond while maintaining contact between the folded absorbent assembly and the apex of the discrete inseam bond; and bonding the plurality of substrates of the folded cover assembly together with side seam bonds.

A boxer-style absorbent article may be produced. The boxer-style absorbent article may comprise a front panel, a back panel, and an absorbent assembly. The front panel and the back panel may be joined at an inseam bond and at a pair of side seam bonds. The absorbent assembly may be positioned between the front panel and the back panel and bonded thereto. The absorbent assembly may be separated from an apex of the inseam bond by a distance of about 0 to about 25 mm.

These and other features, aspects, and advantages of various configurations will become better understood with reference to the following description, figures, and claims.

### BRIEF DESCRIPTION OF THE DRAWINGS

Many aspects of this disclosure can be better understood with reference to the following figures.

FIG. 1 is an example according to various configurations illustrating a top view of a boxer-style absorbent article laid flat.

FIG. 2 is an example according to various configurations illustrating a perspective view of a boxer-style absorbent article.

FIG. 3A is an example according to various configurations illustrating a perspective view of a first substrate and a second substrate in alignment.

FIG. 3B is an example according to various configurations illustrating a perspective view of the first substrate and the second substrate in alignment as shown in FIG. 3A, but with a plurality of elastic strands disposed therebetween.

FIG. 4A is an example according to various configurations illustrating a perspective view of a pre-folded assemblage comprising two sets of substrates aligned along a folding axis.

FIG. 4B is an example according to various configurations illustrating a perspective view of the pre-folded assemblage of FIG. 4A, but with a set of elastic strands disposed between each set of substrates.

FIG. 5A is an example according to various configurations illustrating a perspective view of a pre-folded assemblage comprising three substrates aligned about a folding axis.

FIG. 5B is an example according to various configurations illustrating a perspective view of the pre-folded assemblage of FIG. 5A, but with two sets of elastic strands disposed therebetween.

FIG. 6A is an example according to various configurations illustrating a perspective view of a pre-folded assemblage comprising a pair of substrates aligned with each other and centered along a folding axis.

FIG. 6B is an example according to various configurations illustrating a perspective view of the pre-folded assemblage of FIG. 6A, but with the pair of substrates having two sets of elastic strands disposed therebetween.

FIG. 7 is an example according to various configurations illustrating a perspective view showing the formation of a cover assembly from a first substrate and a second substrate.

FIG. 8 is an example according to various configurations illustrating a perspective view of showing the formation of a cover assembly from a first substrate having a laminate structure and a second substrate having a laminate structure.

FIG. 9A is an example according to various configurations illustrating a perspective view of the formation of shaped holes along a folding axis in one of three substrates shown in FIG. 5A.

FIG. 9B is an example according to various configurations illustrating a perspective view of the formation of slits along a folding axis in one of three substrates shown in FIG. 5A.

FIG. 9C is an example according to various configurations illustrating a top view of a cover assembly formed after folding the three substrates along the folding axis shown in FIG. 9B.

FIG. 10A is an example according to various configurations illustrating a perspective view of the formation of shaped holes along a folding axis in the pair of substrates shown in FIG. 6A.

FIG. 10B is an example according to various configurations illustrating a perspective view of the formation of slits along a folding axis in the pair of substrates shown in FIG. 6A.

FIG. 10C is an example according to various configurations illustrating a top view of a cover assembly formed after folding the pair of substrates along the folding axis shown in FIG. 10B.

FIG. 11A is an example according to various configurations illustrating a top view of a generalized cover assembly.

FIG. 11B is an example according to various configurations illustrating a top view of a generalized cover assembly including processing aid bonds between discrete inseam bonds.

FIG. 12 is an example according to various configurations illustrating a top view of the generalized cover assembly being unfolded along an unfolding axis aligned with a plurality of apexes of a plurality of discrete inseam bonds.

FIG. 13 is an example according to various configurations illustrating a top view of an unfolded segment of the generalized cover assembly.

FIG. 14 is an example according to various configurations illustrating a top view of a plurality of absorbent assemblies placed over the unfolded segment of the generalized cover assembly shown in FIG. 13.

FIG. 15 is an example according to various configurations illustrating a cross-sectional view along line A-A as shown in FIG. 14.

FIG. 16 is an example according to various configurations illustrating a top view of the generalized cover assembly shown in FIG. 14 after being refolded.

FIG. 17 is an example according to various configurations illustrating a cross-sectional view along line B-B as shown in FIG. 16.

FIG. 18 is an example according to various configurations illustrating a top view of the generalized cover assembly shown in FIG. 16, showing formation of side seam bonds, and cutting along the side seam bonds to form a discrete absorbent article.

FIG. 19A is an example according to various configurations illustrating a top view of a boxer-style absorbent article with a discrete inseam bond having a semi-circular shape.

FIG. 19B is an example according to various configurations illustrating a top view of a boxer-style absorbent article with a discrete inseam bond having a triangular shape.

FIG. 19C is an example according to various configurations illustrating a top view of a boxer-style absorbent article with a discrete inseam bond having a rectangular shape.

FIG. 19D is an example according to various configurations illustrating a top view of a boxer-style absorbent article with a discrete inseam bond having a trapezoidal shape.

FIG. 19E is an example according to various configurations illustrating a top view of a boxer-style absorbent article with a discrete inseam bond formed around a cut.

It should be understood that the various configurations are not limited to the examples illustrated in the figures.

### DETAILED DESCRIPTION

This disclosure is written for a person having ordinary skill in the art, who will understand that this disclosure is not limited to the specific examples or configurations described. The examples and configurations are single instances which will make a much larger scope apparent to the person having ordinary skill in the art. Unless defined otherwise, all technical and scientific terms used herein have the same meaning as commonly understood by the person having ordinary skill in the art. It is also to be understood that the terminology used herein is for the purpose of describing examples and configurations only, and is not intended to be limiting, since the scope of the present disclosure will be limited only by the appended claims.

All the features disclosed in this specification (including any accompanying claims, abstract, and drawings) may be replaced by alternative features serving the same, equivalent, or similar purpose, unless expressly stated otherwise. Thus, unless expressly stated otherwise, each feature disclosed is one example only of a generic series of equivalent or similar features. The examples and configurations described herein are for illustrative purposes only and that various modifications or changes in light thereof will be suggested to the person having ordinary skill in the art and are to be included within the spirit and purview of this application. Many variations and modifications may be made to the configurations of the disclosure without departing substantially from the spirit and principles of the disclosure. All such modifications and variations are intended to be included herein within the scope of this disclosure. For example, unless otherwise indicated, the present disclosure is not limited to particular materials, reagents, reaction materials, manufacturing processes, or the like, as such can vary. It is also to be understood that the terminology used herein is for purposes of describing particular configurations only and is not intended to be limiting. It is also possible in the present disclosure that steps can be executed in different sequence where this is logically possible.

All numeric values are herein assumed to be modified by the term “about,” whether or not explicitly indicated. The term “about” generally refers to a range of numbers that one of skill in the art would consider equivalent to the recited value (for example, having the same function or result). In many instances, the term “about” may include numbers that are rounded to the nearest significant figure.

In everyday usage, indefinite articles (like “a” or “an”) precede countable nouns and noncountable nouns almost never take indefinite articles. It must be noted, therefore, that, as used in this specification and in the claims that follow, the singular forms “a,” “an,” and “the” include plural referents unless the context clearly dictates otherwise. Thus, for example, reference to “a support” includes a plurality of supports. Particularly when a single countable noun is listed as an element in a claim, this specification will generally use a phrase such as “a single.” For example, “a single support.”

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

In this specification and in the claims that follow, reference will be made to a number of terms that shall be defined to have the following meanings unless a contrary intention is apparent.

“Disposed on” refers to a positional state indicating that one object or material is arranged in a position adjacent to the position of another object or material. The term does not require or exclude the presence of intervening objects, materials, or layers.

“Disposed between” refers to a positional state indicating that one object or material is arranged in a position intermediate to the position of other objects or materials. The term does not require or exclude the presence of intervening objects, materials, or layers.

“Belt length” refers to a distance measured along a hip portion of a garment from a waist portion to a leg hole.

“Waist-to-inseam length” refers to a distance measured along a longitudinal axis of the garment from the waist to the crotch thereof.

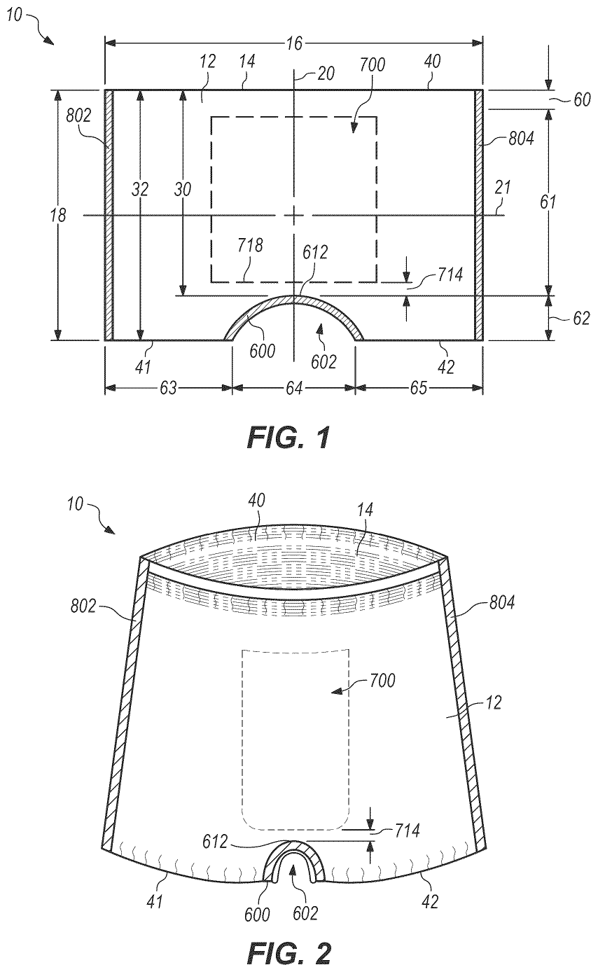

“Boxer-style” refers to a type of diaper or garment that is fundamentally different from a “traditional” pant diaper in at least two ways. First, a boxer-style garment's belt length is longer than its waist-to-inseam length. These lengths are illustrated in FIG. 1. Secondly, a boxer-style garment has an inseam bond and an inseam cut.

“Absorbent article” refers to devices that absorb and contain liquid, and more specifically, refers to devices that are placed against or in proximity to the body of the wearer to absorb and to contain various exudates discharged from the body.

“Align” or “aligned” or “aligning” means to place or to arrange in a straight line. Aligning edges of substrates, therefore, means arranging the substrates so that the edges in question extend along approximately the same line. It is to be appreciated that aligning edges of substrates can be accomplished in a variety of ways, including placing the substrates one on top of the other or side by side.

“Facing relationship” refers to a relative positioning of materials, such as substrates, in which a surface of one material is oriented toward a surface of another material. For example, when two substrates are stacked on top of each other, they are in a facing relationship. The term does not require or exclude the presence of intervening objects, materials, or layers.

“Machine direction” (MD) refers to the direction of material flow through a process. In addition, relative placement and movement of material can be described as flowing in the machine direction through a process from upstream in the process to downstream in the process.

“Cross direction” (CD) refers to a direction that is generally perpendicular to the machine direction.

FIG. 1 is an example according to various configurations illustrating a top view of a boxer-style absorbent article 10 laid flat. The article 10 may comprise a front panel 12 and a back panel 14 joined at a first side seam bond 802, a second side seam bond 804 and an inseam bond 600. The article may comprise several regions along its longitudinal axis 20, including a waist region 60, a leg-crotch region 62 and a body region 61 disposed therebetween. The article may comprise several regions along its lateral axis 21, including a first leg region 63, a second leg region 65, and a crotch region 64 disposed therebetween. The article 10 may have an overall width 16 measured along the longitudinal axis 20 and an overall height 18 measured along the lateral axis 21.

Still referring to FIG. 1, a top opening 40 may be formed in the waist region 60 between the first panel 12, the second panel 14, the first side seam bond 802, and the second side seam bond 804. A first leg hole 41 may be defined in the leg-crotch region 62 and in the first leg region 63 between the first panel 12, the second panel 14, the first side seam bond 802, and the inseam bond 600. A second leg hole 42 may be defined in the leg-crotch region 62 and in the second leg region 65 between the first panel 12, the second panel 14, the second side seam bond 804, and the inseam bond 600. The inseam bond 600 may be formed in the leg-crotch region 62 and the crotch region 64. The inseam bond 600 may have a variety of shapes, as will be discussed herein, but the specific example of an inseam bond 600 that is illustrated in FIG. 1 has a semi-circular shape 602. The inseam bond 600 may have an apex 612. The apex 612 may be a portion of the inseam bond 600 that is closest to the waist region 60 of the article 10. A waist-to-inseam length 30 may be less than a belt length 32 of the absorbent article 10, such that the article 10 is a boxer-style article. The article 10 may further comprise an absorbent assembly 700 disposed between the first panel 12 and the second panel 14. The absorbent assembly 700 may be folded such that a folded portion 718 of the absorbent assembly 700 is positioned at a distance 714 from the apex 612.

It is to be appreciated that the absorbent assembly 700 may be configured in various ways and may include various types of materials. For example, the absorbent assembly 700 may include an absorbent core disposed between a topsheet and a backsheet. The absorbent article 10 may also include other features, such as leg elastics and/or leg cuffs to enhance the fit around the legs of the wearer. The backsheet may comprise a woven or nonwoven material, polymeric films such as thermoplastic films of polyethylene or polypropylene, and/or a multi-layer or composite materials comprising a film and a nonwoven material. The backsheet may also comprise an elastomeric film. An example backsheet may be a polyethylene film having a thickness of from about 0.012 mm (0.5 mils) to about 0.051 mm (2.0 mils). Further, the backsheet may permit vapors to escape from the absorbent core (i.e., the backsheet is breathable) while still preventing exudates from passing through the backsheet. The topsheet may be liquid pervious, permitting liquids (e.g., menses, urine, and/or runny feces) to penetrate through its thickness. A topsheet may be manufactured from a wide range of materials such as woven and nonwoven materials; apertured or hydroformed thermoplastic films; apertured nonwovens, porous foams; reticulated foams; reticulated thermoplastic films; and thermoplastic scrims. Woven and nonwoven materials may comprise natural fibers such as wood or cotton fibers; synthetic fibers such as polyester, polypropylene, or polyethylene fibers; or combinations thereof. If the topsheet includes fibers, the fibers may be spunbond, carded, wet-laid, meltblown, hydroentangled, or otherwise processed as is known in the art. Topsheets may be selected from high loft nonwoven topsheets, apertured film topsheets and apertured nonwoven topsheets. Exemplary apertured films may include those described in U.S. Pat. Nos. 5,628,097; 5,916,661; 6,545,197; and 6,107,539, all of which are incorporated by reference herein. The absorbent core may be formed in various sizes and shapes that are compatible with the absorbent assembly. Exemplary absorbent structures for use as the absorbent core of the present disclosure are described in U.S. Pat. Nos. 4,610,678; 4,673,402; 4,888,231; and 4,834,735, all of which are incorporated by reference herein. Some absorbent core configurations may comprise fluid storage cores that contain reduced amounts of cellulosic airfelt material. For instance, such cores may comprise less than about 40%, 30%, 20%, 10%, 5%, or even 1% of cellulosic airfelt material. Such a core may comprise primarily absorbent gelling material in amounts of at least about 60%, 70%, 80%, 85%, 90%, 95%, or even about 100%, where the remainder of the core comprises a microfiber glue (if applicable). Such cores, microfiber glues, and absorbent gelling materials are described in U.S. Pat. Nos. 5,599,335; 5,562,646; 5,669,894; and 6,790,798 as well as U.S. Patent Publication Nos. 2004/0158212 A1 and 2004/0097895 A1, all of which are incorporated by reference herein. It is to be appreciated that the first panel 12 and the second panel 14 may be configured in various ways and may include various types of materials, such as described above with reference exemplary topsheet and/or backsheet materials.

FIG. 2 is an example according to various configurations illustrating a perspective view of a boxer-style absorbent article 10. FIG. 2 more clearly shows the first panel 12 and the second panel 14, as well as the top opening 40, the first leg hole 41, and the second leg hole 42. The first panel 12 and the second panel 14 may be formed in a variety of ways from a variety of substrates.

The first panel 12 and the second panel 14 may be formed in any suitable manner. An enormous number of variations will be apparent to those having ordinary skill in the art upon reviewing the present disclosure. While several variations are explicitly illustrated and discussed, the present disclosure is not intended to be limited to these examples.

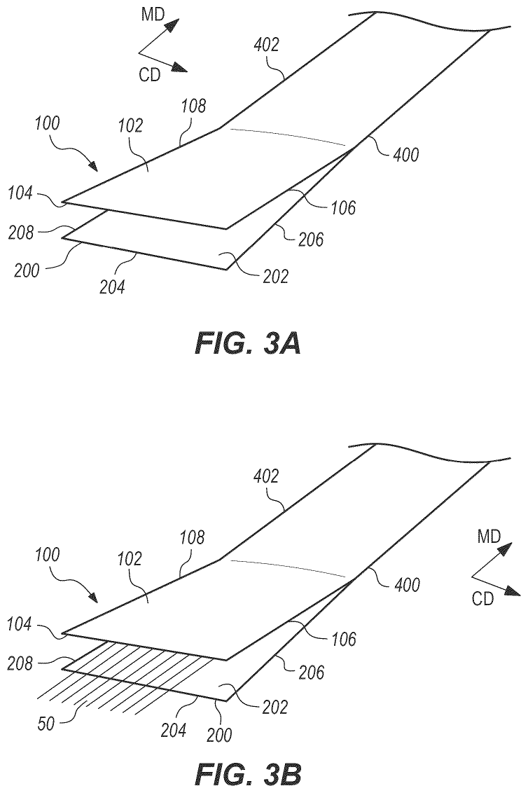

FIG. 3A is an example according to various configurations illustrating a perspective view of a first substrate 100 and a second substrate 200 in alignment. The first substrate 100 may have a first surface 102 and an opposing second surface 104 as well as a first edge 106 and a second edge 108. The first edge 106 and the second edge 108 of the first substrate 100 may be separated in a cross direction CD. Similarly, the first substrate 200 may have a first surface 202 and an opposing second surface 204 as well as a first edge 206 and a second edge 208. The first edge 206 and the second edge 208 of the second substrate 200 may be separated in a cross direction CD. The first substrate 100 and the second substrate 200 may be arranged in a facing relationship such that the second surface 104 of the first substrate 100 is disposed on the first surface 202 of the second substrate 200. The first edge 106 of the first substrate 100 may be aligned with the first edge 206 of the second substrate 200. Similarly, the second edge 108 of the first substrate 100 may be aligned with the second edge 208 of the second substrate 200. The first substrate 100 and the second substrate 200 may be separated by a first unbonded edge region 400 along their first edges 106, 206. The first unbonded edge region 400 may, after additional processing, provide the first leg hole 41 and/or the second leg hole 42 of the article 10. Similarly, the first substrate 100 and the second substrate 200 may be separated by a second unbonded edge region 402 along their second edges 108, 208. The second unbonded edge region 402 may, after additional processing, provide the top opening 40 of the article 10. The aligned substrates 100 and 200 may be advanced in a machine direction MD. It is to be appreciated that according to this configuration, the first panel 12 of an absorbent article 10 may ultimately be formed from the first substrate 100, and the second panel 14 of the absorbent article 10 may ultimately be formed from the second substrate 200.

FIG. 3B is an example according to various configurations illustrating a perspective view of the first substrate 100 and the second substrate 200 in alignment as shown in FIG. 3A, but with elastic material 50, illustrated in the form of a plurality of elastic strands 50, disposed therebetween. The elastic strands 50 may be provided in any configuration and may provide resiliency and flexibility to the article 10. The elastic strands 50 may be bonded to either the first substrate 100 or to the second substrate 200. Additionally or alternatively, the elastic strands 50 may be disposed between layers of a laminated structure, such as is illustrated in FIG. 8. It is also to be appreciated that various types of elastic material may be utilized herein instead of elastic strands or in combination with elastic strands. Such elastic material may include one or more elastic elements such as strands, ribbons, films, or panels. Elastic strands may be configured in various ways and may have various decitex values. In some configurations, the elastic strands may be configured with decitex values ranging from about 10 decitex to about 1000 decitex, specifically reciting all 1 decitex increments within the above-recited range and all ranges formed therein or thereby.

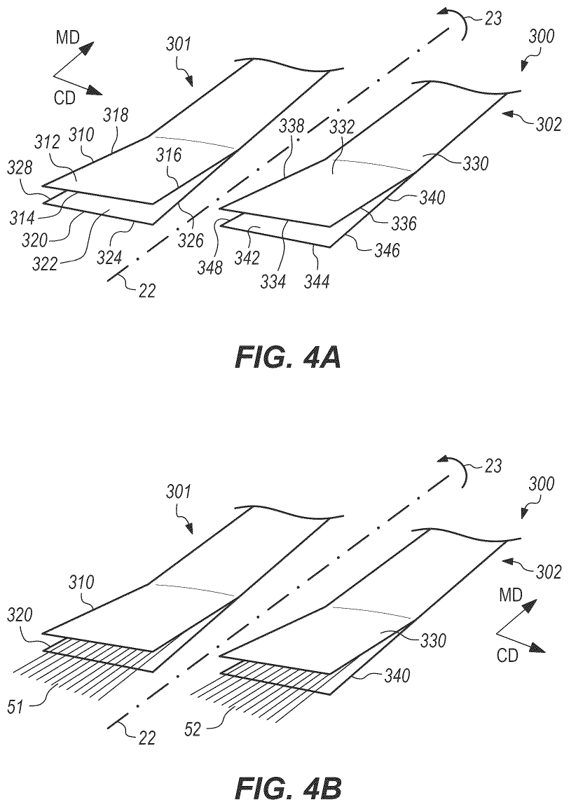

FIG. 4A is an example according to various configurations illustrating a perspective view of a pre-folded assemblage 300. The pre-folded assemblage 300 may comprise a first set of substrates 301 and a second set of substrates 302. The first set of substrates 301 may comprise a first substrate 310 and a second substrate 320. It is to be appreciated that additional substrates, layers, and materials may be present in the first set of substrates 301. The first substrate 310 may have a first surface 312 and an opposing second surface 314, as well as a first edge 316 and an opposing second edge 318. The second substrate 320 may have a first surface 322 and an opposing second surface 324, as well as a first edge 326 and an opposing second edge 328. The first substrate 310 and the second substrate 320 may be positioned in a facing relationship and aligned. Positioning the first substrate 310 and the second substrate 320 in a facing relationship may comprise disposing the first substrate 310 on the second substrate 320 such that the second surface 314 of the first substrate 310 is disposed on the first surface 322 of the second substrate 320. Aligning the first substrate 310 and the second substrate 320 may comprise aligning the first edge 316 of the first substrate 310 with the first edge 326 of the second substrate 320 and/or aligning the second edge 318 of the first substrate 310 with the second edge 328 of the second substrate 320. The second set of substrates 302 may comprise a third substrate 330 and a fourth substrate 340. It is to be appreciated that additional substrates, layers, and materials may be present in the second set of substrates 302. The third substrate 330 may have a first surface 332 and an opposing second surface 334, as well as a first edge 336 and an opposing second edge 338. The fourth substrate 340 may have a first surface 342 and an opposing second surface 344, as well as a first edge 346 and an opposing second edge 348. The third substrate 330 and the fourth substrate 340 may be positioned in a facing relationship and aligned. Positioning the third substrate 330 and the fourth substrate 340 in a facing relationship may comprise disposing the third substrate 330 on the fourth substrate 340 such that the second surface 334 of the third substrate 330 is disposed on the first surface 342 of the fourth substrate 340. Aligning the third substrate 330 and the fourth substrate 340 may comprise aligning the first edge 336 of the third substrate 330 with the first edge 346 of the fourth substrate 340 and/or aligning the second edge 338 of the third substrate 330 with the second edge 348 of the fourth substrate 340.

Still referring to FIG. 4A, the first set of substrates 301 and the second set of substrates 302 may be aligned along a folding axis 22. Folding about the folding axis 22 may be accomplished in a variety of ways. For example, the second set of substrates 302 may be folded in a folding direction 23 about the folding axis 22. It is to be appreciated that the first set of substrates 301 could be folded about the folding axis 22 in a direction opposite the exemplified folding direction 23. Additionally, both the first set of substrates 301 and the second set of substrates 302 could be folded about the folding axis 22 simultaneously. In any case, folding about the folding axis 22 may bring the first set of substrates 301 and the second set of substrates 302 into a facing relationship and into alignment. Bringing the first set of substrates 301 and the second set of substrates 302 into a facing relationship may comprise disposing the first surface 332 of the third substrate on the first surface 312 of the first substrate 310. Aligning the first set of substrates 301 and the second set of substrates 302 may comprise aligning both the first edge 336 of the third substrate 330 and the first edge 346 of the fourth substrate 340 with both the second edge 318 of the first substrate and the second edge 328 of the second substrate 320; and/or aligning both the second edge 338 of the third substrate 330 and the second edge 348 of the fourth substrate 340 with both the first edge 316 of the first substrate and the first edge 326 of the second substrate 320.

FIG. 4B is an example according to various configurations illustrating a perspective view of the pre-folded assemblage of FIG. 4A, but with a set of elastic strands disposed between each set of substrates. More specifically, a first set of elastic strands 51 may be disposed between the first substrate 310 and the second substrate 320; and a second set of elastic strands 52 may be disposed between the third substrate 330 and the fourth substrate 340.

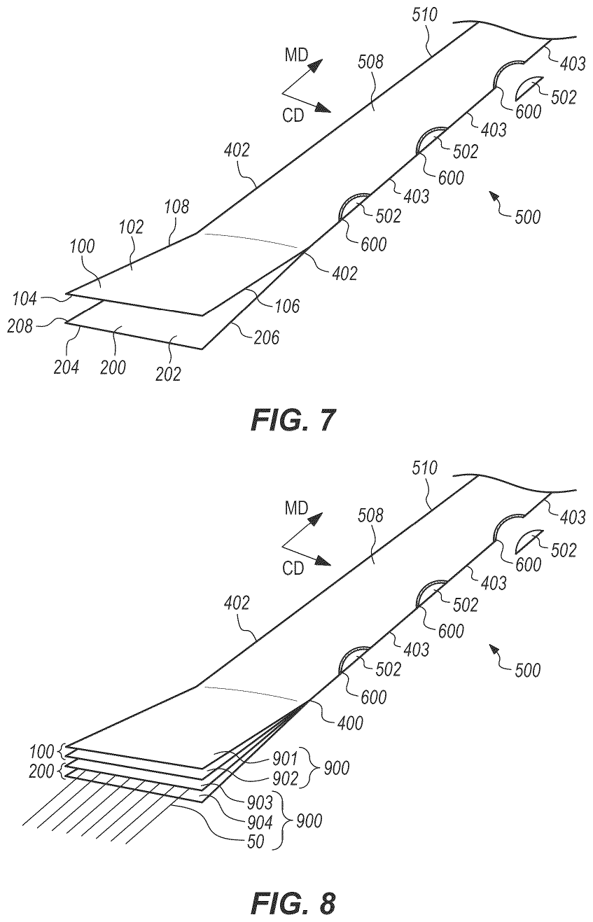

FIG. 4A and FIG. 4B illustrate early steps in a process of forming an absorbent article 10. It is to be appreciated that according to this configuration, the first panel 12 of an absorbent article 10 may ultimately be formed from the second set of substrates 302. Similarly, the second panel 14 of an absorbent article 10 may ultimately be formed from the first set of substrates 301. As will be discussed in greater detail hereinafter, for example, with respect to FIGS. 7, 8, 9C, and 10C, one or more discrete inseam bonds 600 may be formed at the second edge 338 of the third substrate 330, the second edge 348 of the fourth substrate 340, the first edge 316 of the first substrate, and the first edge 326 of the second substrate 320 to join all four substrates 310, 320, 330, 340 together. On the opposite side of the aligned sets of substrates 301, 302, an unbonded edge region 402 may be maintained, which may, after further processing, become the top opening 40 of an absorbent article 10.

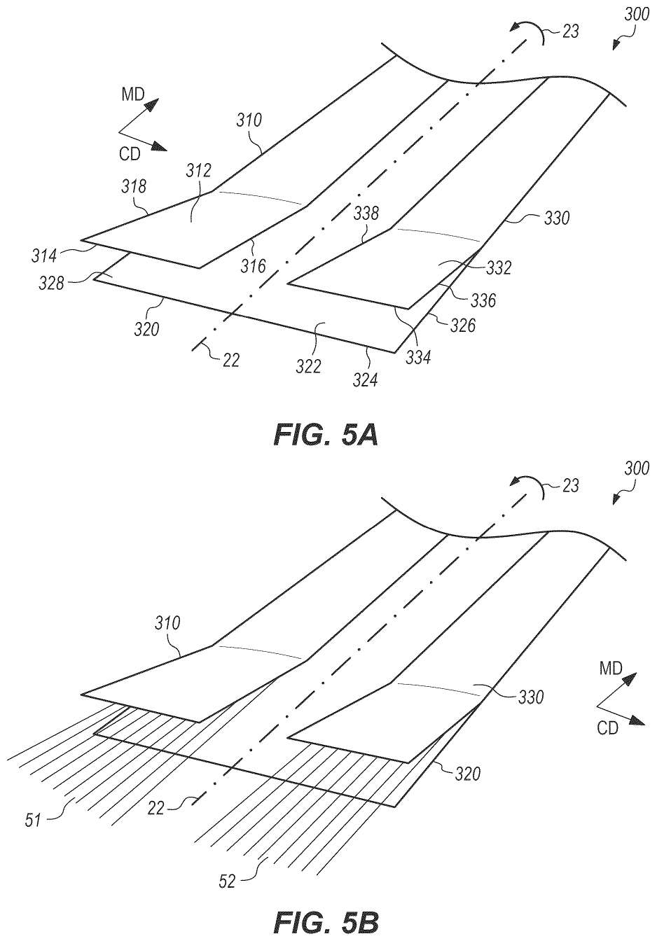

FIG. 5A is an example according to various configurations illustrating a perspective view of a pre-folded assemblage 300 comprising three substrates 310, 320, 330 aligned about a folding axis 22. The pre-folded assemblage 300 may comprise a first substrate 310, a second substrate 320, and a third substrate 330. The first substrate 310 may have a first surface 312 and an opposing second surface 314, as well as a first edge 316 and an opposing second edge 318. The second substrate 320 may have a first surface 322 and an opposing second surface 324, as well as a first edge 326 and an opposing second edge 328. The third substrate 330 may have a first surface 332 and an opposing second surface 334, as well as a first edge 336 and an opposing second edge 338. The second substrate 320 may be wider than the first substrate 310 and the second substrate 320, such that both the first substrate 310 and the third substrate 330 may be positioned in a facing relationship with the second substrate 320 and aligned with the second substrate 320. Positioning the first substrate 310 in a facing relationship with the second substrate 320 may comprise disposing the first substrate 310 on the second substrate 320 such that the second surface 314 of the first substrate 310 is disposed on the first surface 322 of the second substrate 320. Positioning the third substrate 330 in a facing relationship with the second substrate 320 may comprise disposing the third substrate 330 on the second substrate 320 such that the second surface 334 of the third substrate 330 is disposed on the first surface 322 of the second substrate 320. Aligning the first substrate 310 and the second substrate 320 may comprise aligning the second edge 318 of the first substrate 310 with the second edge 328 of the second substate 320. Aligning the third substrate 330 and the second substrate 320 may comprise aligning the first edge 336 of the third substrate 330 with the first edge 326 of the second substate 320. The second substrate 320 may be folded along the folding axis 22, for example in a folding direction 23. Upon folding, the first edge 336 of the third substrate 330 and the first edge 326 of the second substrate 320 may be aligned with the second edge 318 of the first substrate 310 and the second edge 328 of the second substrate 320.

FIG. 5B is an example according to various configurations illustrating a perspective view of the pre-folded assemblage 300 of FIG. 5A, but with two sets of elastic strands 51, 52 disposed therebetween. More specifically, a first set of elastic strands 51 may be disposed between the first substrate 310 and the second substrate 320; and a second set of elastic strands 52 may be disposed between the third substrate 330 and the second substrate 320.

FIG. 5A and FIG. 5B illustrate early steps in a process of forming an absorbent article 10. It is to be appreciated that according to this configuration, the first panel 12 of an absorbent article 10 may ultimately be formed from a combination of the first substrate 310 and a portion of the second substrate 320. Similarly, the second panel 14 of an absorbent article 10 may ultimately be formed from the third substrate and a portion of the second substrate 320. As will be discussed in greater detail hereinafter, for example, with respect to FIGS. 7, 8, 9C, and 10C, after folding, one or more discrete inseam bonds 600 may be formed in the second substrate 320 along the folding axis 22. On the opposite side an unbonded edge region 402 may be maintained between the first substrate 310 and the third substrate 330, which may, after further processing, become the top opening 40 of an absorbent article 10.

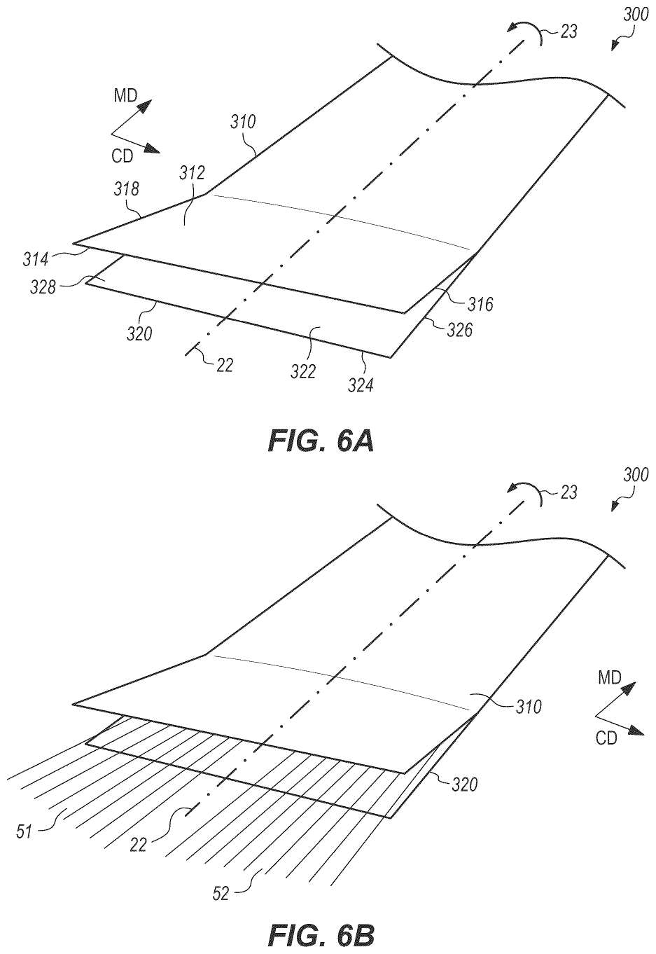

FIG. 6A is an example according to various configurations illustrating a perspective view of a pre-folded assemblage 300 comprising a pair of substrates 310, 320 aligned with each other and centered along a folding axis 22. The first substrate 310 may have a first surface 312 and an opposing second surface 314, as well as a first edge 316 and an opposing second edge 318. The second substrate 320 may have a first surface 322 and an opposing second surface 324, as well as a first edge 326 and an opposing second edge 328. The first substrate 310 and the second substrate 320 may be positioned in a facing relationship and aligned. Positioning the first substrate 310 and the second substrate 320 in a facing relationship may comprise disposing the first substrate 310 on the second substrate 320 such that the second surface 314 of the first substrate 310 is disposed on the first surface 322 of the second substrate 320. Aligning the first substrate 310 and the second substrate 320 may comprise aligning the first edge 316 of the first substrate 310 with the first edge 326 of the second substrate 320 and/or aligning the second edge 318 of the first substrate 310 with the second edge 328 of the second substrate 320. The first substrate 310 and the second substrate 320 may be folded along the folding axis 22, for example in a folding direction 23. Upon folding, the first edge 316 of the first substrate 310 and the first edge 326 of the second substrate 320 may be aligned with the second edge 318 of the first substrate 310 and the second edge 328 of the second substrate 320.

FIG. 6B is an example according to various configurations illustrating a perspective view of the pre-folded assemblage 300 of FIG. 6A, but with the pair of substrates having two sets of elastic strands 51, 52 disposed therebetween. More specifically, a first set of elastic strands 51 may be disposed between the first substrate 310 and the second substrate 320 on one side of the folding axis 22, while a second set of elastic strands 52 may be disposed between the first substrate 310 and the second substrate 320 on the other side of the folding axis 22.

FIG. 6A and FIG. 6B illustrate early steps in a process of forming an absorbent article 10. It is to be appreciated that according to this configuration, the first panel 12 of an absorbent article 10 may ultimately be formed from a portion of the first substrate 310 and a portion of the second substrate 320. Similarly, the second panel 14 of an absorbent article 10 may ultimately be formed from a portion of the first substrate 310 and a portion of the second substrate 320. As will be discussed in greater detail hereinafter, for example, with respect to FIGS. 7, 8, 9C, and 10C, after folding, one or more discrete inseam bonds 600 may be formed in the first substrate 310 and the second substrate 320 along the folding axis 22. On the opposite side an unbonded edge region 402 may be maintained between opposing folded portions of the first substrate 310, which may, after further processing, become the top opening 40 of an absorbent article 10.

Regardless of the specific materials and techniques employed to form the first panel 12 and the second panel 14, a cover assembly 500 may be formed. An enormous number of variations on the layers of a cover assembly 500 as well as how to produce a cover assembly 500 will be apparent to those having ordinary skill in the art upon reviewing the present disclosure. While several variations are explicitly illustrated and discussed, the present disclosure is not intended to be limited to these examples.

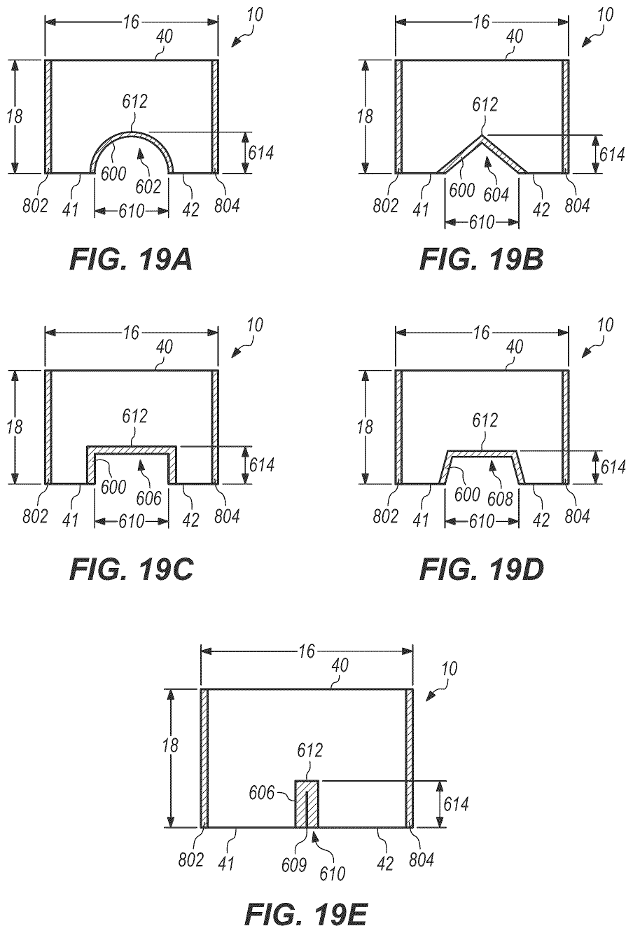

FIG. 7 is an example according to various configurations illustrating a perspective view showing the formation of a cover assembly 500 from a first substrate 100 and a second substrate 200. After being arranged as described with respect to FIGS. 3A and 3B, the substrates 100, 200 may be advanced in a machine direction. One or more inseam bonds 600 may be formed in the substrates 100, 200, joining the substrates 100, 200 to each other to form a cover assembly 500. One or more inseam scrap portions 502 may be cut away from or otherwise removed from the cover assembly 500. The cover assembly 500 may comprise a first panel 508 and a second panel 510. The first panel 508 may comprise the first substrate 100. The second panel 510 may comprise the second substrate 200. The inseam bonds 600 may be spaced, leaving a spacing 403 bounded by the first panel 508, the second panel 510, and a pair of adjacent inseam bonds 600. After further processing, the spacing 403 may be formed into leg holes 41, 42 of an absorbent article 10. Similarly, an unbonded edge region 402 may be maintained between the first panel 508 and the second panel 510 at the opposite edge of the cover assembly 500, which may, after further processing, be formed into a top opening 40 of an absorbent article 10. It is to be appreciated that in some configurations, the cover assembly may include a cut 609 without having a scrap portion removed, such as shown for example in FIG. 19E.

FIG. 8 is an example according to various configurations illustrating a perspective view showing the formation of a cover assembly 500 from a first substrate 100 and a second substrate 200. The first substrate 100 may have a laminate structure 900 comprising a first lamination substrate 901 and a second lamination substrate 902. The second substrate 200 may have a laminate structure 900 comprising a third lamination substrate 903, a plurality of elastic strands, and a fourth lamination substrate 904. It is to be appreciated that any combination of substrates and materials may form a laminate structure. The cover assembly 500 is formed from the first substrate 100 and the second substrate 200 as described with respect to FIG. 7.

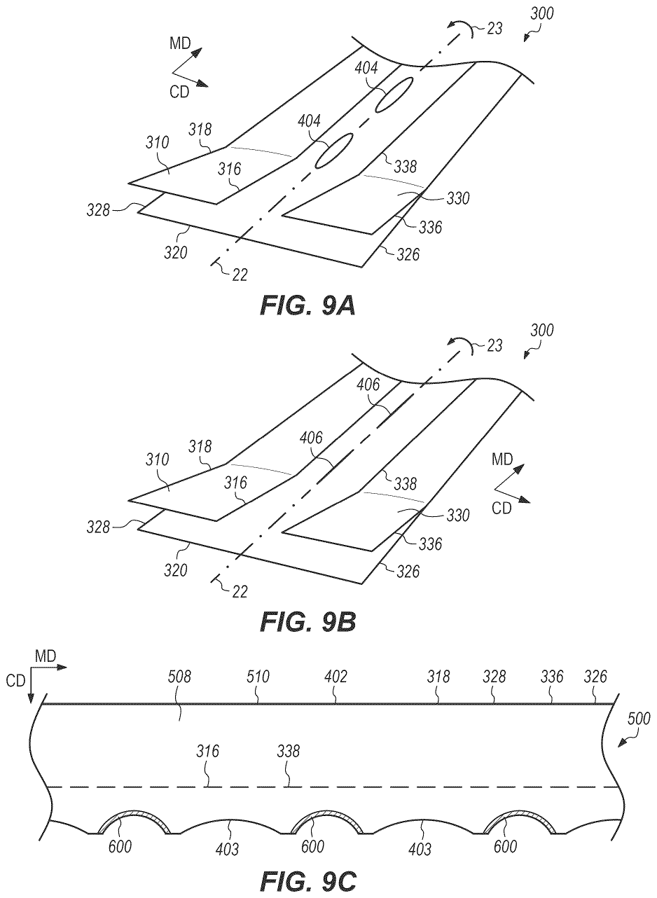

FIG. 9A is an example according to various configurations illustrating a perspective view of the formation of shaped holes 404 along a folding axis 22 in the second substrate 320 shown in FIG. 5A. Similarly, FIG. 9B is an example according to various configurations illustrating a perspective view of the formation of slits 406 along a folding axis 22 in the second substrate 320 shown in FIG. 5A. FIG. 9C is an example according to various configurations illustrating a top view of a cover assembly 500 formed after folding the three substrates 310, 320, 330 along the folding axis 22 shown in FIG. 9A and FIG. 9B.

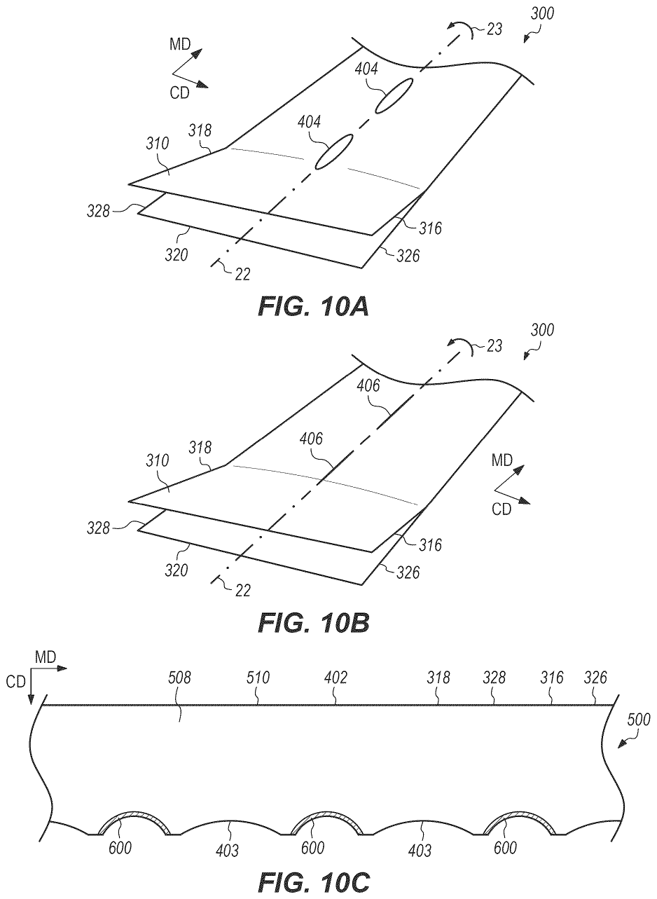

FIG. 10A is an example according to various configurations illustrating a perspective view of the formation of shaped holes 404 along a folding axis 22 in the pair of substrates 310, 320 shown in FIG. 6A. Similarly, FIG. 10B is an example according to various configurations illustrating a perspective view of the formation of slits 406 along a folding axis 22 in the pair of substrates 310, 320 shown in FIG. 6A. FIG. 10C is an example according to various configurations illustrating a top view of a cover assembly 500 formed after folding the pair of substrates 310, 320 along the folding axis shown in FIG. 10A and FIG. 10B.

With reference to either FIG. 9C or FIG. 10C, forming the cover assembly 500 may comprise forming inseam bonds 600 between the shaped holes 404 or the slits 406, which may provide spacings 403 403, which, after further processing, may form leg holes 41, 42 in an absorbent article 10. The cover assembly 500 comprises a first panel 508 and a second panel 510. The first panel 508 may comprise the third substrate 330 and a portion of the second substrate 320. The second panel 510 may comprise the first substrate 310 and a portion of the second substrate 320. An unbonded edge region 402 may be maintained between the first panel and the second panel along an edge opposing the spacings 403. The unbonded edge region 402 may, after further processing, form a top opening 40 of an absorbent article 10.

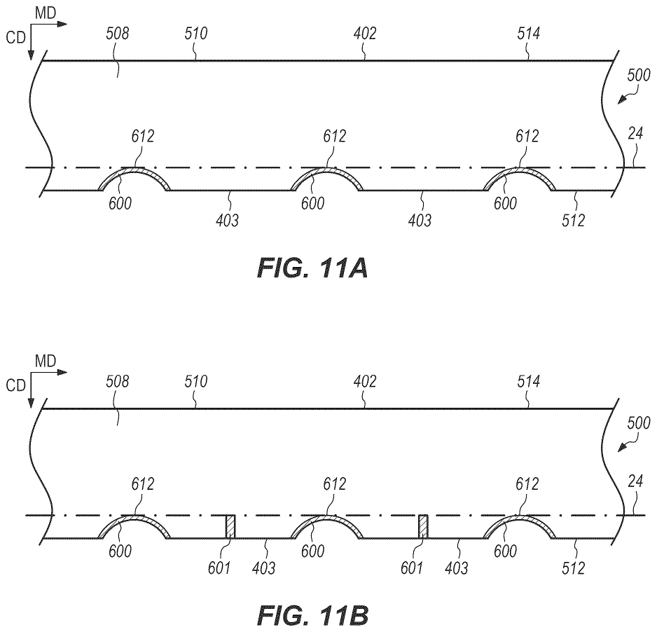

FIG. 11A is an example according to various configurations illustrating a top view of a generalized cover assembly 500. The generalized cover assembly 500 may comprise a first panel 508 and a second panel 510 formed by any of the methods, materials, and techniques described herein. The cover assembly may comprise one or more of inseam bonds 600 along an inseam edge 512 thereof. The cover assembly 500 may comprise spacings 403 on either side of any given inseam bond 600 along the inseam edge 512, as well as an unbonded edge region 402 on the opposing edge 514 of the cover assembly 500. The cover assembly 500 may further comprise an unfolding axis 24 aligned with the apexes 612 of the inseam bonds 600 and parallel to the inseam edge 512. It is to be appreciated that if only a single inseam bond 600 is present, the unfolding axis may intersect with an apex 612 of the single inseam bond 600 and may also be parallel to the inseam edge 512. Methods according to various configurations may comprise producing a cover assembly 500 according to any of the methods and with any of the configurations described herein.

FIG. 11B is an example according to various configurations illustrating a top view of a generalized cover assembly 500, identical to that shown in FIG. 11A, but with processing aid bonds 601 between discrete inseam bonds 600. According to such configurations, simultaneous to, prior to, or following the creation of the inseam bond and/or the inseam cut, a processing aid bond 601 may be added between consecutive inseam bonds. The processing aid bond 601 may help maintain control of the leg hole opening locations during web advancement and subsequent process operations. The processing aid bond may be generated with any typical bonding technologies known to the art including, but not limited to thermal bonding, hot air or through-air bonding, ultrasonic bonding, laser bonding/welding, friction welding, pressure bonding, embossing or mechanical entanglement, infrared heating and bonding, and combinations thereof. Additionally or alternatively, processing aid bonds 601 may be formed via any typical adhesive-based technologies known in the art including, but not limited to hot melt adhesives, pressure sensitive adhesives, pure polymer application, cohesive application, infrared or ultraviolet activated adhesives and additives, and combinations thereof. The processing aid bonds 601 may also be a combination of mechanical and adhesive bond technologies.

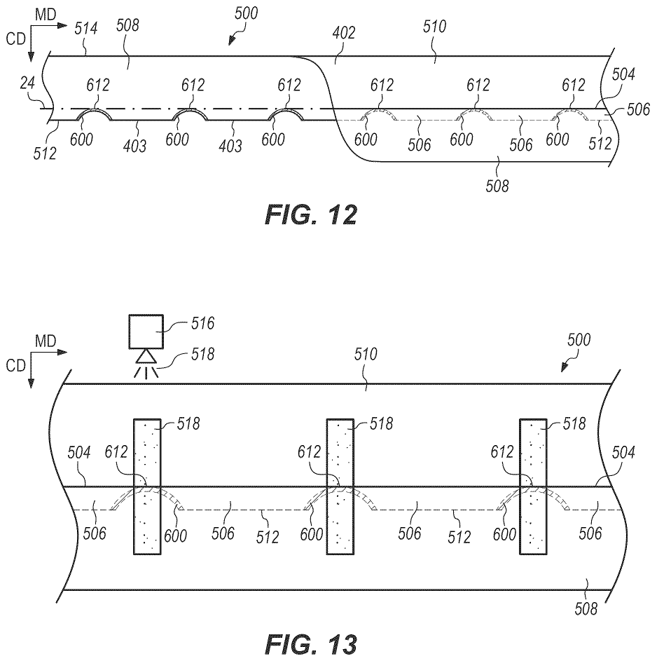

FIG. 12 is an example according to various configurations illustrating a top view of the generalized cover assembly 500 of FIG. 11A being unfolded along the unfolding axis 24 that is aligned with a plurality of apexes 612 of a plurality of discrete inseam bonds 600. The generalized cover assembly 500 may be unfolded by any suitable method or with any suitable equipment. Due to the position of the unfolding axis 24 at the apexes of the inseam bonds, after being unfolded, the cover assembly 500 may comprise an overlapping region 506 at which the first panel and the second panel overlap. The overlapping region 506 may be bounded by a fold 504 along the unfolding axis 24, the inseam edge, and at least one inseam bond.

FIG. 13 is an example according to various configurations illustrating a top view of an unfolded segment of the generalized cover assembly 500 downstream of the point at which the cover assembly 500 is unfolded. The cover assembly 500 may be rolled or otherwise laid flat, ensuring that the first panel 508 and the second panel 510 are laid relatively flat and that the fold 504 is clean and straight. As will be discussed hereinafter, an absorbent assembly 700 may be disposed on the unfolded cover assembly 500. It is to be understood that a bonding composition 518 such as glue may be added to the unfolded cover assembly 500 and/or to the absorbent assembly 700. The bonding composition may be applied on the to the cover assembly 500 as illustrated in FIG. 13 by a bonding composition applicator 516. The bonding composition 518 may be applied in any suitable pattern. It is to be appreciated that the bonding compositions applicator devices 516 herein may be configured in various ways, such as for example, spray nozzles and/or slot coating devices. In some configurations, the adhesive bonding composition applicator devices 516 may be configured in accordance with the apparatuses and/or methods disclosed in U.S. Pat. Nos. 8,186,296; 9,265,672; 9,248,054; and 9,295,590 and U.S. Patent Publication No. 2014/0148773 A1, all of which are incorporated by reference herein.

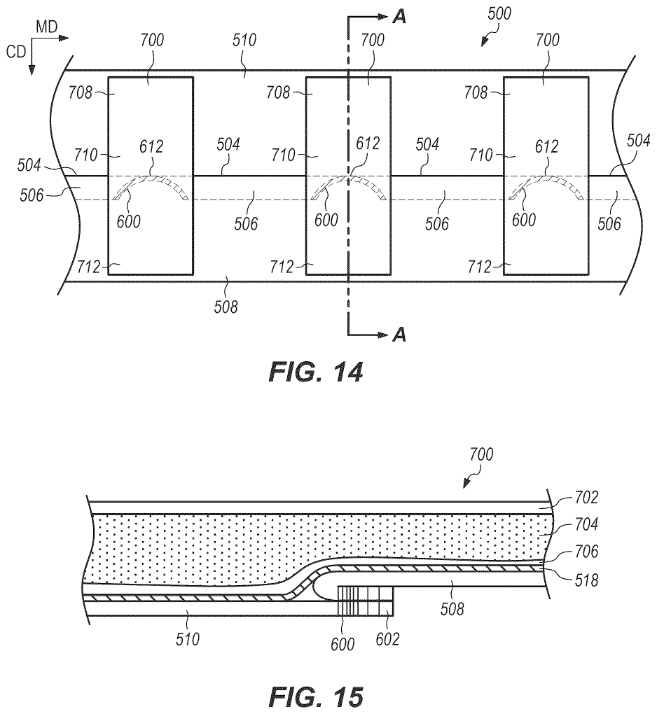

FIG. 14 is an example according to various configurations illustrating a top view of a plurality of absorbent assemblies 700 placed over the unfolded segment of the generalized cover assembly 500 shown in FIG. 13. Bonding composition 518 may be additionally or alternatively applied to the absorbent assemblies 700 prior to disposing the absorbent assemblies 700 on the cover assembly 500. The absorbent assemblies 700 may comprise a first end region 708, a second end region 712, and a crotch region 710 disposed therebetween. The crotch region 710 of each absorbent assembly 700 may be disposed on the fold 504 at the apex 612 of an inseam bond 600.

FIG. 15 is an example according to various configurations illustrating a cross-sectional view along line A-A as shown in FIG. 14. The absorbent assembly 700 may comprise one or more layers. For example, the absorbent assembly 700 may comprise a topsheet 702, a backsheet 706, and an absorbent core 704 disposed therebetween.

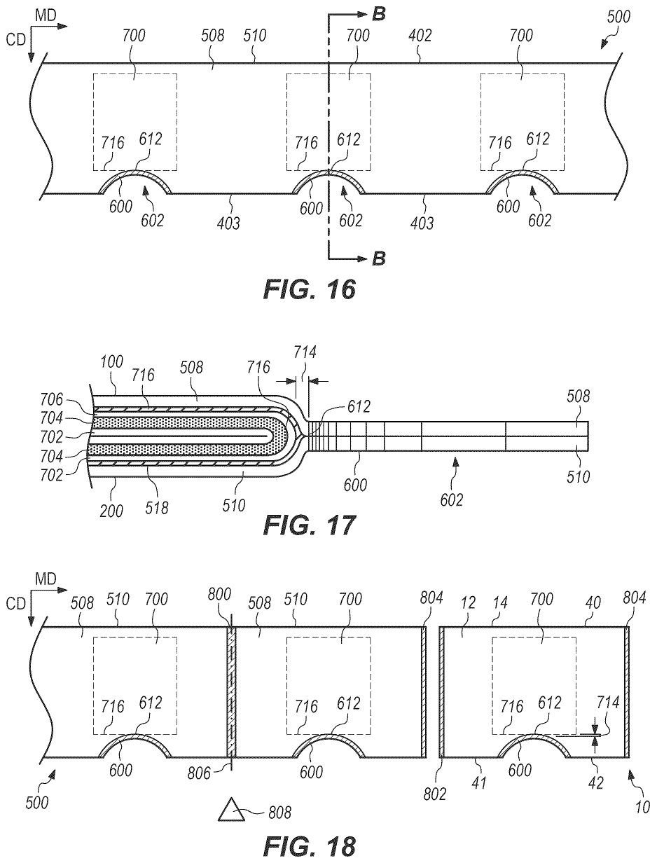

FIG. 16 is an example according to various configurations illustrating a top view of the generalized cover assembly 500 shown in FIG. 14 after being refolded. Once the desired number of absorbent assemblies 700 are correctly positioned at and affixed to the apex 612, the cover assembly 500 maybe refolded. Refolding the cover assembly 500 may comprise folding the absorbent assembly 700 such that the absorbent assembly 700 has a folded portion 716.

FIG. 17 is an example according to various configurations illustrating a cross-sectional view along line B-B as shown in FIG. 16. The absorbent assembly 700 maybe dispersed very near to the apex 612, such that the absorbent assembly 700 is a distance 714 from the apex 612 of the discrete inseam bond 600 to which it was affixed. According to various configurations, the distance 714 may be about 0 to about 25 mm, or about 0 to about 5 mm, or about 0 to about 4 mm, or about 0 to about 3 mm, or about 0 to about 2 mm, or about 0 to about 1 mm.

FIG. 18 is an example according to various configurations illustrating a top view of the generalized cover assembly 500 shown in FIG. 16, showing formation of side seam bonds 800, 802, 804, and cutting along the side seam bonds 800, 802, 804, to form a discrete absorbent article 10. A plurality of uncut side seam bonds 800 may be formed on the cover assembly 500, for example between the applied absorbent assemblies 700. The uncut side seam bonds 800 may be cut along a side seam cutting axis 806 by any suitable method or technique, for example with a cutting apparatus 808. cutting the cover assembly 500 along the uncut side seam bonds 800 may result in a discrete absorbent article 10 having a first side seam bond 802 and a second side seam bond 804.

The inseam bonds 600 may have any suitable shape. FIG. 19A is an example according to various configurations illustrating a top view of a boxer-style absorbent article 10 with a discrete inseam bond 600 having a semi-circular shape 602. FIG. 19B is an example according to various configurations illustrating a top view of a boxer-style absorbent article 10 with a discrete inseam bond 600 having a triangular shape 604. FIG. 19C is an example according to various configurations illustrating a top view of a boxer-style absorbent article 10 with a discrete inseam bond 600 having a rectangular shape 606. FIG. 19D is an example according to various configurations illustrating a top view of a boxer-style absorbent article 10 with a discrete inseam bond 600 having a trapezoidal shape 608. FIG. 19E is an example according to various configurations illustrating a top view of a boxer-style absorbent article 10 with a discrete inseam bond 600 formed around a cut 609. According to various configurations, the discrete inseam bond 600 may have a width 610 that may be about 0 to about 5%, or about 5 to about 45%, or about 10 to about 40%, or about 15 to about 35%, or about 20 to about 30% of a width 16 of the absorbent article 10. According to various configurations, the discrete inseam bond 600 has an apex 612 located at a distance 614 from the second edge 108 of the first substrate 100 and the first edge 206 of the second substrate 200. The distance 614 may be about 1 to about 45%, or about 10 to about 40%, or about 15 to about 35%, or about 20 to about 30% of a height 18 of the absorbent article 10.

Having described each figure individually, a non-limiting description of various configurations will now be provided, making reference to all figures simultaneously.

Various configurations relate to a method for assembling an absorbent article 10. The method may comprise advancing a first substate 100 in a machine direction MD, the first substrate 100 comprising a first surface 102 and an opposing second surface 104, and a first edge 106 separated from a second edge 108 in a cross direction CD. The method may further comprise advancing a second substate 200 in the machine direction MD, the second substrate 200 comprising a first surface 202 and an opposing second surface 204, and a first edge 206 separated from a second edge 208 in a cross direction CD. The method may further comprise positioning the second surface 104 of the first substrate 100 in a facing relationship with the first surface 202 of the second substrate 200. According to various configurations, the at least one of the first substrate 100 and the second substrate 200 comprises a laminate structure 900. The method may further comprise forming a cover assembly 500 by bonding the first substate 100 and the second substrate 200 together adjacent the first edge 106 of the first substrate 100 and the first edge 206 of the second substrate 200 with a discrete inseam bond 600. The method may further comprise unfolding the cover assembly 500 along the discrete inseam bond 600 such that the second edge 108 of the first substrate 100 is separated from the second edge 208 of the second substrate 200 in the cross direction CD. The method may further comprise positioning an absorbent assembly 700 on the unfolded cover assembly 500 and over the inseam bond 600. The method may further comprise folding the cover assembly 500 and absorbent assembly 700 along the inseam bonds 600 to position the second surface 104 of the first substrate 100 in a facing relationship with the first surface 202 of the second substrate 200. The method may further comprise bonding the first substrate 100 and the second substrate 200 of the folded cover assembly 500 together with side seam bonds 800 on opposing sides of the folded absorbent assembly 500. Finally, the method may comprise cutting the folded cover assembly 500 along the side seam bonds 800 to form a discreate absorbent article 10.

According to various configurations, the absorbent assembly 700 may comprises a topsheet 702, a backsheet 706, and absorbent core 704 positioned between the topsheet 702 and the backsheet 706. The absorbent assembly 700 may comprise a first end region 708 and a second end region 712 separated from the first end region 708 by a crotch 710 region. The step of positioning the absorbent assembly 700 on the unfolded cover assembly 500 may comprise positioning the crotch region 710 over the inseam bond 600. The absorbent assembly 700 may be bonded to the absorbent article 10 over the discrete inseam bond 600 in a position providing contact between the absorbent assembly 700 and the discrete inseam bond 600.

According to various configurations, the discrete inseam bond 600 may have a semi-circular shape 602, a triangular shape 604, a rectangular shape 606, or a trapezoidal shape 608. The discrete inseam bond 600 may have a width 610 that is about 10 to about 40% of a width 16 of the absorbent article 10. The discrete inseam bond 600 has an apex 612 located at a distance 614 from the second edge 108 of the first substrate 100 and the first edge 206 of the second substrate 200, wherein the distance 614 is about 10 to about 40% of a height 18 of the absorbent article 10. The discrete inseam bond 600 and/or the side seam bonds 800, 802, 804 may be formed via one selected from thermal bonding, hot air bonding, through-air bonding, ultrasonic bonding, laser bonding, laser welding, friction welding, pressure bonding, embossing, mechanical entanglement, infrared heating and bonding, adhesive bonding, and combinations thereof.

Various configurations relate to a boxer-style absorbent article 10 comprising a front panel 12, a back panel 14, and an absorbent assembly 700. The front panel 12 and the back panel 14 may be joined at an inseam bond 600 and at a pair of side seam bonds 800. The absorbent assembly 700 may be positioned between the front panel 12 and the back panel 14 and bonded thereto. The absorbent assembly 700 may be separated from an apex 612 of the inseam bond 600 by a distance 714 of about 0 to about 5 mm. According to various configurations, the inseam bond 600 may have a semi-circular shape 602, a triangular shape 604, a rectangular shape 606, or a trapezoidal shape 608. According to various configurations, at least one of the front panel 12 and the back panel 14 may comprise a laminate structure 900. The inseam bond 600 and/or the side seam bonds 800, 802, 804 may be formed via one selected from thermal bonding, hot air bonding, through-air bonding, ultrasonic bonding, laser bonding, laser welding, friction welding, pressure bonding, embossing, mechanical entanglement, infrared heating and bonding, adhesive bonding, and combinations thereof.

Various configurations relate to a method for producing a boxer-style absorbent article 10. The method may comprise bonding a plurality of substrates with a discrete inseam bond 600 to form a cover assembly 500; cutting along the discrete inseam bond 600 to separate an inseam scrap portion 502 from the cover assembly 500; unfolding the cover assembly 500 at an apex 612 of the discrete inseam bond 600; bonding an absorbent assembly 700 to the unfolded cover assembly 500 over the discrete inseam bond 600 in a position providing contact between the absorbent assembly 700 and the apex 612 of the discrete inseam bond 600; refolding the cover assembly 500 and the absorbent assembly 700 at the apex 612 of the discrete inseam bond 600 while maintaining contact between the folded absorbent assembly 700 and the apex 612 of the discrete inseam bond 600; and bonding the plurality of substrates of the folded cover assembly 500 together with side seam bonds 800.

According to various configurations, the method may further comprise cutting the folded cover assembly 500 along the side seam bonds 800. The method may further comprise folding the plurality of substrates along a folding axis 22 prior to bonding the plurality of substrates with the discrete inseam bond 600. The method may further comprise cutting the plurality of substrates in a plurality of locations along the folding axis 22.

“Unfolding” of the cover assembly 500, for example as shown in FIG. 12, may be an important transformation step to help deliver product benefits, for example, by keeping the product benefit of keeping the inseam bond 600 and absorbent assembly 500 co-located. This unfolding may be performed via various techniques, methods, and apparatuses, including but not limited to helical folding boards/plows; static rails and guides; combinations of folding boards/plows and static rails and guides; combinations of folding boards and vacuum conveyors; holding the first web at the inseam apex line along a line of vacuum holes on a conveyor; and holding the first and second webs at and below the seam apex line between static rails or guides, and folding the second web away from the first web along the static rail/guide edge. Combinations of folding boards and vacuum conveyors may include holding one of the web surfaces on a vacuum conveyor, while unfolding the other via means of helixed components including, but not limited to idlers arranged in a helix; driven rolls arranged in a helix; helical vacuum conveyor; and/or roller elements in the shape of a twist or helix. Holding the first web at the inseam apex line along a line of vacuum holes on a conveyor may include separating the webs first via static guides/rails and/or unfolding the second web along the inseam apex line being held along a line of vacuum holes on a conveyor using: a series of roller or idler components forming a helix; a series of driven roller components forming a helix; a second vacuum conveyor where the belt path forms a helix; and/or a series of static rails/guides whose surfaces form a helical path. Holding the first and second webs at and below the seam apex line between static rails or guides, and folding the second web away from the first web along the static rail/guide edge may be performed using static folding boards or plows; helical static folding boards or plows; angled idler or roller components; and/or angled driven roller components.

The inseam bonds 600 and/or the side seam bonds 800, 802, 804 may be generated with any typical bonding technologies known to the art including, but not limited to thermal bonding, hot air or through-air bonding, ultrasonic bonding, laser bonding/welding, friction welding, pressure bonding, embossing or mechanical entanglement, infrared heating and bonding, and combinations thereof. Additionally or alternatively, inseam bonds 600 and/or the side seam bonds 800, 802, 804 may be formed via any typical adhesive-based technologies known in the art including, but not limited to hot melt adhesives, pressure sensitive adhesives, pure polymer application, cohesive application, infrared or ultraviolet activated adhesives and additives, and combinations thereof. The inseam bonds 600 and/or the side seam bonds 800, 802, 804 may also be a combination of mechanical and adhesive bond technologies.

It is to be appreciated that various process aids may also be utilized in some configurations. For example, simultaneous to, prior to, or following the creation of the inseam bond and/or the inseam cut, a processing aid bond 601 may be added between consecutive inseam bonds, such as shown in FIG. 11B.

Attaching the absorbent assembly 700 to the cover assembly 500 may be achieved via any typical adhesive-based technology known in the art including, but not limited to hot melt adhesives, pressure sensitive adhesives, pure polymer application, cohesive application, infrared or ultraviolet activated adhesives and additives, and combinations thereof. These adhesive applications can be continuous or intermittent, of various shapes and sizes, and of differing and varying application amount/basis weight. Additionally or alternatively, the absorbent assembly 700 may be attached to the cover assembly 500 via known bonding technologies including, but not limited to thermal bonding, hot air or through-air bonding, ultrasonic bonding, laser bonding/welding, friction welding, pressure bonding, embossing or mechanical entanglement, infrared heating and bonding, and combinations thereof. The absorbent assembly 700 may be attached to the cover assembly 500 may be a combination of known mechanical and adhesive bonding technologies.

### Combinations

A1. A method for assembling an absorbent article, the method comprising steps of: advancing a first substate in a machine direction, the first substrate comprising a first surface and an opposing second surface, and a first edge separated from a second edge in a cross direction; advancing a second substate in the machine direction, the second substrate comprising a first surface and an opposing second surface, and a first edge separated from a second edge in a cross direction; positioning the second surface of the first substrate in a facing relationship with the first surface of the second substrate; forming a cover assembly by bonding the first substate and the second substrate together adjacent the first edge of the first substrate and the first edge of the second substrate with a discrete inseam bond; unfolding the cover assembly along the discrete inseam bond such that the second edge of the first substrate is separated from the second edge of the second substrate in the cross direction; positioning an absorbent assembly on the unfolded cover assembly and over the inseam bond; folding the cover assembly and absorbent assembly along the inseam bonds to position the second surface of the first substrate in a facing relationship with the first surface of the second substrate; bonding the first substrate and the second substrate of the folded cover assembly together with side seam bonds on opposing sides of the folded absorbent assembly; and cutting the folded cover assembly along the side seam bonds to form a discreate absorbent article.

B1. The method of paragraph A1, wherein the absorbent assembly comprises a topsheet, a backsheet, and absorbent core positioned between the topsheet and the backsheet.

C1. The method of either paragraph A1 or B1, wherein the absorbent assembly comprises a first end region and a second end region separated from the first end region by a crotch region.

D1. The method of paragraph C1, wherein the step of positioning the absorbent assembly on the unfolded cover assembly further comprises positioning the crotch region over the inseam bond.

E1. The method of any of paragraphs A1-D1, wherein at least one of the first substrate and the second substrate comprises a laminate structure.

F1. The method of any of paragraphs A1-E1, wherein the discrete inseam bond has a semi-circular shape, a triangular shape, a rectangular shape, or a trapezoidal shape.

G1. The method of any of paragraphs A1-F1, wherein the discrete inseam bond has a width that is about 5 to about 45% of a width of the absorbent article.

H1. The method of any of paragraphs A1-G1, wherein the discrete inseam bond has an apex located at a distance from the second edge of the first substrate and the first edge of the second substrate, wherein the distance is about 1 to about 40% of a height of the absorbent article.

I1. The method of any of paragraphs A1-H1, wherein the discrete inseam bond is formed via one selected from the group consisting of thermal bonding, hot air bonding, through-air bonding, ultrasonic bonding, laser bonding, laser welding, friction welding, pressure bonding, embossing, mechanical entanglement, infrared heating and bonding, adhesive bonding, and combinations thereof.

J1. The method of any of paragraphs A1-I1, wherein the absorbent assembly is bonded to the absorbent article over the discrete inseam bond in a position providing contact between the absorbent assembly and the discrete inseam bond.

K1. The method of any of paragraphs A1-J1, wherein side seam bonds are formed via one selected from the group consisting of thermal bonding, hot air bonding, through-air bonding, ultrasonic bonding, laser bonding, laser welding, friction welding, pressure bonding, embossing, mechanical entanglement, infrared heating and bonding, adhesive bonding, and combinations thereof.

A2. A boxer-style absorbent article comprising: a front panel, a back panel, and an absorbent assembly, wherein the front panel and the back panel are joined at an inseam bond and at a pair of side seam bonds, wherein the absorbent assembly is positioned between the front panel and the back panel and bonded thereto, wherein the absorbent assembly is separated from an apex of the inseam bond by a distance of about 0 to about 25 mm.

B2. The article of paragraph A2, wherein the inseam bond has a semi-circular shape, a triangular shape, a rectangular shape, or a trapezoidal shape.

C2. The article of either paragraph A2 or B2, wherein at least one of the front panel and the back panel comprises a laminate structure.

D2. The article of any of paragraphs A2-C2, wherein the inseam bond is formed via one selected from the group consisting of thermal bonding, hot air bonding, through-air bonding, ultrasonic bonding, laser bonding, laser welding, friction welding, pressure bonding, embossing, mechanical entanglement, infrared heating and bonding, adhesive bonding, and combinations thereof.

E2. The article of any of paragraphs A2-D2, wherein side seam bonds are formed via one selected from the group consisting of thermal bonding, hot air bonding, through-air bonding, ultrasonic bonding, laser bonding, laser welding, friction welding, pressure bonding, embossing, mechanical entanglement, infrared heating and bonding, adhesive bonding, and combinations thereof.

A3. A method for producing a boxer-style absorbent article, the method comprising: bonding a plurality of substrates with a discrete inseam bond to form a cover assembly; cutting along the discrete inseam bond to separate an inseam scrap portion from the cover assembly; unfolding the cover assembly at an apex of the discrete inseam bond; bonding an absorbent assembly to the unfolded cover assembly over the discrete inseam bond in a position providing contact between the absorbent assembly and the apex of the discrete inseam bond; refolding the cover assembly and the absorbent assembly at the apex of the discrete inseam bond while maintaining contact between the folded absorbent assembly and the apex of the discrete inseam bond; and bonding the plurality of substrates of the folded cover assembly together with side seam bonds.

B3. The method of paragraph A3, further comprising cutting the folded cover assembly along the side seam bonds.

C3. The method of either paragraph A3 or B3, further comprising folding the plurality of substrates along a folding axis prior to bonding the plurality of substrates with the discrete inseam bond.

D3. The method of any of paragraphs A3-C3, further comprising cutting the plurality of substrates in a plurality of locations along the folding axis.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present disclosure have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method for assembling an absorbent article, the method comprising steps of:
advancing a first substate in a machine direction, the first substrate comprising a first surface and an opposing second surface, and a first edge separated from a second edge in a cross direction;
advancing a second substate in the machine direction, the second substrate comprising a first surface and an opposing second surface, and a first edge separated from a second edge in a cross direction;
positioning the second surface of the first substrate in a facing relationship with the first surface of the second substrate;
forming a cover assembly by bonding the first substate and the second substrate together adjacent the first edge of the first substrate and the first edge of the second substrate with a discrete inseam bond;
unfolding the cover assembly along the discrete inseam bond such that the second edge of the first substrate is separated from the second edge of the second substrate in the cross direction;
positioning an absorbent assembly on the unfolded cover assembly and over the inseam bond;
folding the cover assembly and absorbent assembly along the inseam bonds to position the second surface of the first substrate in a facing relationship with the first surface of the second substrate;
bonding the first substrate and the second substrate of the folded cover assembly together with side seam bonds on opposing sides of the folded absorbent assembly; and
cutting the folded cover assembly along the side seam bonds to form a discreate absorbent article.

2. The method of claim 1, wherein the absorbent assembly comprises a topsheet, a backsheet, and absorbent core positioned between the topsheet and the backsheet.

3. The method of claim 1, wherein the absorbent assembly comprises a first end region and a second end region separated from the first end region by a crotch region.

4. The method of claim 3, wherein the step of positioning the absorbent assembly on the unfolded cover assembly further comprises positioning the crotch region over the inseam bond.

5. The method of claim 1, wherein at least one of the first substrate and the second substrate comprises a laminate structure.

6. The method of claim 1, wherein the discrete inseam bond has a semi-circular shape, a triangular shape, a rectangular shape, or a trapezoidal shape.

7. The method of claim 1, wherein the discrete inseam bond has a width that is about 5 to about 45% of a width of the absorbent article.

8. The method of claim 1, wherein the discrete inseam bond has an apex located at a distance from the second edge of the first substrate and the first edge of the second substrate, wherein the distance is about 1 to about 40% of a height of the absorbent article.

9. The method of claim 1, wherein the discrete inseam bond is formed via one selected from the group consisting of thermal bonding, hot air bonding, through-air bonding, ultrasonic bonding, laser bonding, laser welding, friction welding, pressure bonding, embossing, mechanical entanglement, infrared heating and bonding, adhesive bonding, and combinations thereof.

10. The method of claim 1, wherein the absorbent assembly is bonded to the absorbent article over the discrete inseam bond in a position providing contact between the absorbent assembly and the discrete inseam bond.

11. The method of claim 1, wherein side seam bonds are formed via one selected from the group consisting of thermal bonding, hot air bonding, through-air bonding, ultrasonic bonding, laser bonding, laser welding, friction welding, pressure bonding, embossing, mechanical entanglement, infrared heating and bonding, adhesive bonding, and combinations thereof.

12. An absorbent article comprising:
a front panel, a back panel, and an absorbent assembly, wherein the front panel and the back panel are joined at an inseam bond and at a pair of side seam bonds;
wherein the absorbent assembly is positioned between the front panel and the back panel and bonded thereto; and
wherein the absorbent assembly is separated from an apex of the inseam bond by a distance of about 0 to about 25 mm.

13. The article of claim 12, wherein the inseam bond has a semi-circular shape, a triangular shape, a rectangular shape, or a trapezoidal shape.

14. The article of claim 12, wherein at least one of the front panel and the back panel comprises a laminate structure.

15. The article of claim 12, wherein the inseam bond is formed via one selected from the group consisting of thermal bonding, hot air bonding, through-air bonding, ultrasonic bonding, laser bonding, laser welding, friction welding, pressure bonding, embossing, mechanical entanglement, infrared heating and bonding, adhesive bonding, and combinations thereof.

16. The article of claim 12, wherein side seam bonds are formed via one selected from the group consisting of thermal bonding, hot air bonding, through-air bonding, ultrasonic bonding, laser bonding, laser welding, friction welding, pressure bonding, embossing, mechanical entanglement, infrared heating and bonding, adhesive bonding, and combinations thereof.

17. A method for producing an absorbent article, the method comprising:
bonding a plurality of substrates with a discrete inseam bond to form a cover assembly;
cutting along the discrete inseam bond to separate an inseam scrap portion from the cover assembly;
unfolding the cover assembly at an apex of the discrete inseam bond;
bonding an absorbent assembly to the unfolded cover assembly over the discrete inseam bond in a position providing contact between the absorbent assembly and the apex of the discrete inseam bond;
refolding the cover assembly and the absorbent assembly at the apex of the discrete inseam bond while maintaining contact between the folded absorbent assembly and the apex of the discrete inseam bond; and
bonding the plurality of substrates of the folded cover assembly together with side seam bonds.

18. The method of claim 17, further comprising cutting the folded cover assembly along the side seam bonds.

19. The method of claim 17, further comprising folding the plurality of substrates along a folding axis prior to bonding the plurality of substrates with the discrete inseam bond.

20. The method of claim 17, further comprising cutting the plurality of substrates in a plurality of locations along the folding axis.
